(19) United States
(12) Patent Application Publication
(10) Pub. No.: US 2017/0165282 A1
(43) Pub. Date: Jun. 15, 2017

(54) USE OF BETA-NICOTINAMIDE MONONUCLEOTIDE IN PREPARATION OF DRUGS FOR PREVENTING AND TREATING ARTERIOSCLEROSIS AND CARDIO-CEREBROVASCULAR DISEASES, AND DRUGS CONTAINING THE SAME

(71) Applicant: BONTAC BIO-ENGINEERING (SHENZHEN) CO., LTD, Shenzhen (CN)

(72) Inventors: Rongzhao FU, Shenzhen (CN); Yanyan CAI, Shenzhen (CN)

(73) Assignee: BONTAC BIO-ENGINEERING (SHENZHEN) CO., LTD., Shenzhen (CN)

(21) Appl. No.: 15/317,848

(22) PCT Filed: Dec. 11, 2015
(86) PCT No.: PCT/CN2015/097072

§ 371 Date: Dec. 9, 2016

(51) Int. Cl. A61K 31/706; A61K 9/00

(52) U.S. Cl. CPC A61K 31/706; A61K 9/0053

(57) ABSTRACT

A medication having nicotinamide mononucleotide as the active ingredient, and the use of nicotinamide mononucleotide in preparing medications for the prevention and treatment of arteriosclerosis and cardiovascular and cerebrovascular diseases caused by arteriosclerosis.

### BACKGROUND

Technical Field

The present invention relates to the technical field of use of β-nicotinamide mononucleotide, and particularly to use of β-nicotinamide mononucleotide in biomedicine.

Related Art

Arteriosclerosis is a non-inflammatory lesion of arteries, which causes the walls of arteries to thicken, harden and lose elasticity, and narrows the artery lumen. Arteriosclerosis mainly includes arteriolar sclerosis, medial calcinosis, and atherosclerosis, where the atherosclerosis is the most common arteriosclerosis, and is a main cause of myocardial infarction and cerebral infarction.

Arteriosclerosis is a vascular disease which occurs with the increase of human's age. Regularly, arteriosclerosis usually takes place in adolescence, and is aggravated in middle-old age, and the incidence in male is higher than in female. In recent years, the incidence of the disease gradually rises in China, and has become one of the main causes of death in the elderly. Atherosclerosis is also a major cause of cardiovascular disease which is in turn a common disease seriously threatening human health, especially in the middle-aged to elderly people over the age of 50 years. Even in the case that the most advanced and perfect treatment is used, there are still over 50% of cerebrovascular accident survivors that can not take care of themselves independently. The annual mortality caused by cardio-cerebrovascular diseases is up to 15 million people in the whole world, and cardio-cerebrovascular diseases are the first leading cause of death among others. Therefore, there is a need for seeking a powerful drug for preventing and treating arteriosclerosis, so as to stay away from the cardiovascular disease and maintain a healthy body.

Pathogenesis of atherosclerosis is complex, and is a result of long-term cooperative actions of various factors. Drugs currently used to treat arteriosclerosis are mostly statin lipid-lowering drugs, such as simvastatin, pravastatin, lovastatin and so on. It is well known that these chemically synthesized drugs often have a high toxic side effect, and will cause great harm to human body when administered at a high dose or for a long period of time.

For example, the State Food and Drug Administration issued a warning for simvastatin that after administration, unexpected myalgia, haphalgesia and weakness may occur, and rhabdomyolysis may occur in a severe case, which is much worse for patients. In addition, these statin lipid-lowering drugs are generally expensive.

β-nicotinamide mononucleotide (NMN) is a biochemical substance present in biological cells, which plays an important role in the energy production of human cells. It participates in the intracellular synthesis of nicotinamide adenine dinucleotide (NAD, an important coenzyme for cell energy conversion), and is harmless to human body. At present, the research on use of β-nicotinamide mononucleotide in biomedicine has been carried out, and the use of β-nicotinamide mononucleotide in the anti-aging and treatment of Parkinson's disease has been reported. However, no report about the use of β-nicotinamide mononucleotide in treating arteriosclerosis is found.

### SUMMARY

In order to solve the technical problems of large toxic effects and high price of drugs for treating arteriosclerosis mentioned in the background, the present invention provides new use of β-nicotinamide mononucleotide in the field of biomedicine, and particularly use of β-nicotinamide mononucleotide in the prevention and treatment of arteriosclerosis and cardio-cerebrovascular diseases resulting therefrom. It is anticipated to obtain an inexpensive, safe, and effective drug for preventing and treating arteriosclerosis and cardio-cerebrovascular diseases resulting therefrom.

To achieve the above objective, long-term intensive research on the use of β-nicotinamide mononucleotide in medicine is carried out by the inventors, and it is found surprisingly that β-nicotinamide mononucleotide is effective in preventing and treating arteriosclerosis. Therefore, the present invention provides use of β-nicotinamide mononucleotide in the preparation of drugs for preventing and treating arteriosclerosis and cardio-cerebrovascular diseases resulting therefrom. β-nicotinamide mononucleotide.

At present, β-nicotinamide mononucleotide can be produced through fermentation with yeasts or chemical synthesis, or produced enzymatically in vitro. The β-nicotinamide mononucleotide used in the present invention is produced enzymatically in vitro. The β-nicotinamide mononucleotide produced enzymatically in vitro is advantageous in that the purity is high, no organic solvent residue is present, no problem of chirality occurs, and the prepared product is an isoform of the β-NMN in organisms. Therefore, when used as a drug for preventing and treating arteriosclerosis, it has the benefits of fast onset of action, high therapeutic effect, stable efficacy, and harmlessness to human. Furthermore, the β-nicotinamide mononucleotide prepared as such is inexpensive, which can largely reduce related consumer expenditure.

The β-nicotinamide mononucleotide, as an active ingredient, may be processed with pharmaceutically acceptable different adjuvants into various pharmaceutical dosage forms by using conventional preparation processes, for example, granules, powders, tablets, pills, capsules, oral liquid, and injections, for being administered to the patients. Because the route of oral administration is convenient and less harmful to human body, the drug is preferably administered orally.

Depending on the disease condition, age, body weight, personal traits, and others of the patients, the dosage of β-nicotinamide mononucleotide administered varies greatly. For an adult, the β-nicotinamide mononucleotide is preferably administered at a dosage of 0.1-20 mg/kg body weight/day.

Preferably, the drug further contains nicotinamide riboside.

Similar to β-nicotinamide mononucleotide, nicotinamide riboside (NR) is also a biochemical substance present in biological cells, which is a precursor of vitamin B₃ and nicotinamide adenine dinucleotide, and plays an important role in the energy production of human cells. It is found by the inventors through research that when a small amount of nicotinamide riboside is used in combination with β-nicotinamide mononucleotide as main active ingredient, the effect of preventing and treating arteriosclerosis can be improved to some extent. Likewise, the nicotinamide riboside used in the present invention is also produced enzymatically in vitro.

More preferably, the nicotinamide riboside is administered at a dosage that is 0.1-0.5 time of that of the β-nicotinamide mononucleotide.

Further, the present invention also provides a drug for preventing and treating arteriosclerosis and cardio-cerebrovascular diseases resulting therefrom, comprising β-nicotinamide mononucleotide as active ingredient.

Preferably, the drug is an oral preparation, which can be prepared into various pharmaceutical dosage forms by using conventional preparation processes, for example, granules, powders, tablets, pills, capsules, and oral liquid.

Preferably, the β-nicotinamide mononucleotide is administered at a dosage of 0.1-20 mg/kg body weight/day.

Preferably, the drug further comprises nicotinamide riboside.

More preferably, the nicotinamide riboside is administered at a dosage that is 0.1-0.5 time of that of the β-nicotinamide mononucleotide.

### Beneficial Effects

In the present invention, β-nicotinamide mononucleotide is initially used as an active ingredient in the preparation of drugs for preventing and treating arteriosclerosis and cardio-cerebrovascular diseases resulting therefrom. β-nicotinamide mononucleotide and nicotinamide riboside are both biochemical substances present in biological cells. Such substances produced in vivo have not only a good effect for preventing and treating arteriosclerosis, but also no toxic effects, and thus have an extremely high safety. It is confirmed by animal experiments that β-nicotinamide mononucleotide, as an active ingredient, is quite effective in preventing the occurrence of and treating arteriosclerosis, and the effect is even better when the β-nicotinamide mononucleotide is adjuvanted with a small amount of nicotinamide riboside. It is confirmed by clinical trials that one month after orally taking β-nicotinamide mononucleotide, the total effective rate in patients having cardio-cerebrovascular diseases resulting from arteriosclerosis can be up to 96%; one month after orally taking both the β-nicotinamide mononucleotide and the nicotinamide riboside, the total effective rate can be further up to 98%; and one month after intramuscularly injecting β-nicotinamide mononucleotide and nicotinamide riboside, the total effective rate can be up to 99%.

Moreover, the β-nicotinamide mononucleotide used in the present invention is produced enzymatically in vitro, and the β-nicotinamide mononucleotide produced enzymatically in vitro is advantageous in that the purity is high, no organic solvent residue is present, no problem of chirality occurs, and the prepared product is an isoform of the β-NMN in organisms. Therefore, when used as a drug for preventing and treating arteriosclerosis and cardio-cerebrovascular diseases resulting therefrom, it has the benefits of fast onset of action, high therapeutic effect, stable efficacy, and harmlessness to human. Furthermore, the β-nicotinamide mononucleotide prepared as such is inexpensive, which can largely reduce related consumer expenditure.

### DETAILED DESCRIPTION

The present invention is described in further detail below with reference to specific examples. The examples below are illustrative of the present invention, and the present invention is not limited thereto.

β-nicotinamide mononucleotide (NMN) and nicotinamide riboside (NR) in examples below are both produced enzymatically in vitro.

### Example 1

### Data of Animal Experiment

### 1. Experimental Method:

80 SPF healthy guinea pigs (female:male 1:1) having a body weight of 250-300 g, were assigned to 8 groups at random, each group having 10 animals. The groups include:

(1) a normal control group, fed on basal diet, and allowed to free access to water during experiment;

(2) a high fat control group, fed on high-fat diet, and allowed to free access to water during experiment;

(3) a high fat prevention group, fed on high-fat diet, dosed with 10 mg NMN/kg body weight by gavage every day, and allowed to free access to water during experiment;

(4) a low-dose NMN group, fed on high-fat diet, dosed with 0.1 mg NMN/kg body weight by gavage every day from week 7, and allowed to free access to water during experiment;

(5) a medium-dose NMN group, fed on high-fat diet, dosed with 10 mg NMN/kg body weight by gavage every day from week 7, and allowed to free access to water during experiment;

(6) a high-dose NMN group, fed on high-fat diet, dosed with 20 mg NMN/kg body weight by gavage every day from week 7, and allowed to free access to water during experiment;

(7) NMN+NR gavage group: fed on high-fat diet, dosed with 10 mg NMN/kg body weight and 1 mg NR/kg body weight by gavage every day from week 7, and allowed to free access to water during experiment.

(8) NMN+NR injection group: fed on high-fat diet, dosed with 10 mg NMN/kg body weight and 5 mg NR/kg body weight by intramuscular injection every day from week 7, and allowed to free access to water during experiment.

The formulation of the high-fat diet includes 83% basal diet, 2% cholesterol, and 15% lard oil.

At the end of week 10, all the experimental animals was fasted (allowed to access to water) for 12 hrs and then sacrificed. The aorta was isolated, dissected longitudinally along the midline of the anterior wall, and stained with Sudan IV. The lesion of the aorta was observed with naked eyes. The lesion area pathologically measured and the total endangium area were quantified, and the degree of arteriosclerosis was expressed by percentages of the lesion area relative to the total endangium area of the aorta.

Statistical analysis: All the experimental data is analyzed by SPSS13.0 Statistics software. The measurement data is expressed as x̅±s, and t test is applied to check the statistical significance. Where P<0.05, there is statistical significance.

### 2. Experimental Result

It is observed that the aorta of the animals in the normal control group has smooth and intact endangium free of lesions. The endangium of the aorta of the animals in the group fed on high-fat diet is thickened and coarsened, and has round or irregular yellow plaque or fatty streak of needle tip to mung bean size protruded from the surface thereof. However, the lesions to the aorta in the high fat control group are more serious than those of the animals in other gavage groups. The quantitative data of the lesions to the aorta of the animals in each group is shown in Table 1.

TABLE 1

| Group | Rate of lesion area of aorta (%) |
|---|---|
| (1) Normal control group | 0 |
| (2) High fat control group | 71 ± 3 |
| (3) High fat prevention group | 13 ± 2 |
| (4) Low-dose NMN group | 58 ± 5 |
| (5) Medium-dose NMN group | 27 ± 3 |
| (6) High-dose NMN group | 18 ± 4 |
| (7) NMN + NR gavage group | 23 ± 1 |
| (8) NMN + NR injection group | 20 ± 2 |

### Example 2

### Clinical Data Trial

### 1. Selection of Patients

300 patients (male:female 1:1, aged 35-78 years and 57.5 years on average, and having a course of disease of the shortest 1.5 years, the longest 7 years, and 4.6 years on average) diagnosed as having cardio-cerebrovascular diseases resulting from arteriosclerosis were selected at random as subjects for clinical observation. The patients were assigned to a group A, a group B, and a group C at random, each group having 100 patients.

### 2. Administration Method

Group A: The patients were orally administered with NMN at a dosage of 10 mg/kg body weight before or after meals every day, and the diet was controlled as desired.

Group B: The patients were orally administered with NMN at a dosage of 10 mg/kg body weight and with NR at a dosage of 2 mg/kg body weight before or after meals every day, and the diet was controlled as desired.

Group C: The patients were intramusculary injected with NMN at a dosage of 10 mg/kg body weight and with NR at a dosage of 3 mg/kg body weight before or after meals every day, and the diet was controlled as desired.

### 3. Criteria for Efficacy Evaluation

(1) Obvious effectiveness: The health is built up, the body is powerful, the blood lipid and blood pressure drop to normal levels, the palpitation, chest pain, chest tightness, headache, dizziness, decreased vision, decreased memory, insomnia and dreamful sleep, and other clinical symptoms are obvious alleviated.

(2) Effectiveness: The health is built up, the blood lipid and blood pressure drop slightly, the palpitation, chest pain, chest tightness, headache, dizziness, decreased vision, decreased memory, insomnia and dreamful sleep, and other clinical symptoms are alleviated to some extent.

(3) Ineffectiveness: There is no obvious improvement in the clinical symptoms.

### 4. Clinical Trial Result

Group A: The patients with mild disease are cured 7 days after administration. After 1 month, obvious effectiveness is shown in 67 out of 100 patients involved in the trial, effectiveness is shown in 28 patients, ineffectiveness is shown in 4 patients, and 1 patient withdraws from the trial. Excluding the withdrawing patients, the total effective rate is 96%.

Group B: The patients with mild disease are cured 5 days after administration. After 1 month, obvious effectiveness is shown in 73 out of 100 patients involved in the trial, effectiveness is shown in 25 patients, and ineffectiveness is shown in 2 patients. The total effective rate is 98%.

Group C: The patients with mild disease are cured 3 days after administration. After 1 month, obvious effectiveness is shown in 81 out of 100 patients involved in the trial, effectiveness is shown in 17 patients, and ineffectiveness is shown in 1 patients. The total effective rate is 99%.

What is claimed is:

1. Use of β-nicotinamide mononucleotide in the preparation of drugs for preventing and treating arteriosclerosis and cardio-cerebrovascular diseases resulting therefrom.

2. The use according to claim 1, wherein the β-nicotinamide mononucleotide is produced enzymatically in vitro.

3. The use according to claim 1, wherein the β-nicotinamide mononucleotide is administered at a dosage of 0.1-20 mg/kg body weight/day.

4. The use according to claim 1, wherein the drug further comprises nicotinamide riboside (NR).

5. The use according to claim 4, wherein the nicotinamide riboside is administered at a dosage that is 0.1-0.5 time of that of the β-nicotinamide mononucleotide.

6. A drug for preventing and treating arteriosclerosis and cardio-cerebrovascular diseases resulting therefrom, comprising β-nicotinamide mononucleotide as an active ingredient.

7. The drug according to claim 6, wherein the drug is an oral preparation.

8. The drug according to claim 6, wherein the β-nicotinamide mononucleotide is administered at a dosage of 0.1-20 mg/kg body weight/day.

9. The drug according to claim 6, further comprising nicotinamide riboside (NR).

10. The drug according to claim 9, wherein the nicotinamide riboside is administered at a dosage that is 0.1-0.5 time of that of the β-nicotinamide mononucleotide.
